(19) United States
(12) Patent Application Publication
(10) Pub. No.: US 2008/0243032 A1
(43) Pub. Date: Oct. 2, 2008

(54) ANALYTICAL DEVICE INCLUDING STERILE PROTECTION

(76) Inventors: Fritz Hindelang, Carlsberg (DE); Karin Schwind, Schifferstadt (DE)

Correspondence Address: ROCHE DIAGNOSTICS OPERATIONS INC., 9115 Hague Road, Indianapolis, IN, 46250-0457, US

(21) Appl. No.: 12/105,304

(22) Filed: Apr. 18, 2008

Related U.S. Application Data
(63) Continuation of application No. PCT/EP2006/009944, filed on Oct. 14, 2006.

(30) Foreign Application Priority Data
Oct. 20, 2005 (EP) 05 022 830.3

(51) Int. Cl. A61B 5/157

(52) U.S. Cl. 600/583

(57) ABSTRACT

An analytical device is provided comprising a lancet and a test element in an integrated arrangement. The lancet generally comprises a lancet needle and a protective cap wherein the lancet needle can be displaced relative to the protective cap and wherein the protective cap generally completely surrounds the tip of the lancet needle. The protective cap is typically comprised of an elastic or plastic material and is configured to generally seal an opening provided in a chamber of the test element. In certain embodiments, the lancet comprises a conductive material and can be configured for electrical connection to a detection and evaluation device in order to be used to measure the filling level of the sample liquid and/or to serve as a counter or reference electrode. The present invention also relates to a process for producing such an analytical device and the use thereof.

### CLAIM OF PRIORITY

The present application is a continuation application based on and claiming priority to PCT/EP2006/009944, filed Oct. 14, 2006, which claims priority to European Patent Application No. 05 022 830.3, filed Oct. 20, 2005, each of which are hereby incorporated by reference in their entireties.

### TECHNICAL FIELD OF THE INVENTION

The present invention relates generally to an analytical device, and more particularly to an analytical device comprising a lancet and an analytical test chamber, wherein one or both of the lancet and the test chamber are protected in a sterile manner. The present invention further relates to a process for producing such an analytical device.

### BACKGROUND

In clinical diagnostics the examination of blood, samples enables an early and reliable detection of pathological conditions as well as the specific and accurate monitoring of physical conditions. Medical blood diagnostics often requires the collection of a blood sample from the individual to be examined. Whereas several milliliters of blood are collected by venipuncture from a person to be examined in hospitals and in the case of physicians in private practice in order lo be able to carry out a plurality of laboratory tests on the blood, nowadays from a few microliters to less than 1 microliter of blood is often sufficient for individual analyses that are directed towards one particular parameter. Such small amounts of blood do not typically require a complicated and painful venipuncture. Rather in such cases it is sufficient to push a sterile, sharp lancet for example into the finger pad or the earlobe of the person to be examined in order to collect blood through the skin and thus to obtain a few microliters or even less for the analysis. This method is especially suitable when the blood sample can be analyzed directly after the blood collection.

Lancets and suitable devices therefor (so-called blood collection devices, blood lancet devices or lancing aids) are available which enable a collection of blood that is as pain-free as possible, especially in the field of so-called “home monitoring” i.e. where medical laymen carry out simple analyses of the blood themselves. A common example is the regular blood collection by diabetics that has to be carried out several times daily to monitor the blood glucose concentration. Furthermore the use of lancets with lancing aids should lower the psychological threshold when lancing one's own body. This is of special importance particularly for children that suffer from diabetes and have to rely on regular blood glucose tests. Examples of lancets and lancing aids are the commercially available devices (lancing aids) and lancets Glucolet® from Bayer AG and Softclix® from Roche Diagnostics GmbH. Such lancets and devices (lancing aids) are for example the subject matter of WO-A 98/48695, EP-A 0 565 970, U.S. Pat. No. 4,442,836 or U.S. Pat. No. 5,554,166, the disclosures of which are hereby incorporated herein by reference in their entireties.

The home monitoring for self-determination of blood sugar is nowadays a common method that is used worldwide for diabetes monitoring. Blood sugar devices in the prior art such as e.g. ACCU-CHEK® Advantage (from Roche, Diagnostics) consist of a measuring device into which a test element (also known as a dispo, test strip or sensor) is inserted. The test strip is brought into contact with a drop of blood which was previously obtained from the finger pad or another part of the body by means of a lancet and lancing aid. The numerous system components (lancet, lancing aid, test strip and measuring device) require much space and result in a relatively complex handling. Meanwhile there are also systems with a higher degree of integration and thus a simpler handling. These for example include the ACCU-CHEK® Compact (from Roche Diagnostics), the Glucometer Dex (from Bayer Diagnostics) and the SofTact (from Medisense). In the case of the two first-mentioned systems, the test strips are stored in a magazine and provided for measurement in the measuring device.

A next step in system simplification can for example be achieved by integrating several functions or functional elements in a single analytical device (disposable). The operating process can for example be considerably simplified by a suitable combination of the lancing process and sensory analyte concentration detection on a test strip. Examples of this can be found in the prior art, including EP 0 199 484 B1, U.S. Pat. No. 6,143,164, and U.S. Pat. No. 4,627,445, the disclosures of which are hereby incorporated by reference herein in their entireties.

The integration of test elements has in addition resulted in the development of new protective elements for the lancets. Since the use of the lancet in an integrated system proceeds in a different manner than in the case of lancets that are inserted manually, a method must be found for keeping the lancet sterile but nevertheless available at any time. The removal of a sterile protection just before use by the user, which is common practice for lancets that are inserted manually, is no longer necessary for integrated systems. Exemplary solutions for this also can be found in the prior art, including US Patent Application Publication Nos. 2003/153939 and 2003/050573, the disclosures of which are hereby incorporated by reference herein in their entireties.

A shortcoming of known dispos is the lack of integration of the various functions such as sterile protection of the lancet, sterile protection of the test space, and shortest possible distances for the blood transport from an application site or opening to a testing area, without the patient having to intervene. Thus, although there are solutions for the individual aspects, there is no solution for the combination of all these requirements. Thus, for example in the prior art in the case of a sealing of housing openings, the sealing material is pierced, and because it is not possible to move the sealing material, an additional opening has to be generated in order to take up the sample. As a result a complicated structure which provides a spatial separation of the lancing process and blood collection is necessary for integrated systems which use sterile protection. Consequently the patient is forced to interact in the blood withdrawal process. This means that there is a major loss in comfort and it is very complicated, especially for patients with impaired sight.

In view of the disadvantages of the prior art, it is an object of the present invention to provide analytical devices (also referred to as “test elements”, “test strips”, “disposables” or “dispos”, depending on the configuration of the devices) which do not have the disadvantages of the prior art. For example, lancet sterility should be ensured by a dispo according to the present invention for the period it is in use while at the same time ensuring a useful integration of the lancet and test element. In this regard, the shortest possible distance for the sample liquid to advance from advance from the dispo opening to the test element should be ensured. This system should have a high degree of operating convenience for the patient due to the fact that the patient does not have to concern himself further with blood collection after the lancing.

It is a further object of the present invention to provide analytical devices with lancets in which at least the lancet needle tip is sterile in the unused state until directly before use, which generally means that it is kept free of germs and bacteria, and can then be hygienically stored in the used state. Ideally this object should be achieved without the user having to employ separate measures for the hygienic storage. Moreover, the user should be protected from accidental injury with the lancet and in particular the used lancet. Finally it should be possible to simply transfer the sample from the site of blood collection to the site of blood examination.

These objects and others that will be apparent to those of ordinary skill in the art are achieved by the embodiments of the present invention as disclosed herein and as may be characterized in the claims attached hereto.

### SUMMARY

In a first embodiment of the present invention, an analytical device is provided which contains a lancet, the lancet typically comprising a lancet needle having a tip and a protective cap which generally completely surrounds the lancet needle at least in the area of the tip. The lancet needle can be displaced relative to the protective cap in at least one direction. The protective cap can comprise various materials that can be penetrated by the lancet and in which the tip of the lancet needle can be embedded. The lancet needled is configured to engage the protective cap after displacement of the needle in the at least one direction and, upon displacement in another direction, to pull the protective cap therealong. The analytical device additionally comprises an analytical test element comprising a chamber which contains a detection element with a reagent system. This chamber includes an opening through which the lancet is moved during the lancing process. As will be described in more detail, this opening can closed by the protective cap of the lancet needle.

Finally, a process for producing such analytical devices is a further aspect of the present invention.

The invention is to be explained in more detail by the following figures and examples.

### BRIEF DESCRIPTION OF THE DRAWINGS

The following detailed description of the embodiments of the present invention can be best understood when read in conjunction with the following drawings, where like structure is indicated with like reference numerals and in which:

FIGS. 1a-1c show a sequence in a side-view of a dispo according to the present invention in an unused state, while in action, and in the measuring state with 2 electrodes, respectively.

FIG. 1d shows a top-view of a dispo according to the present invention in an unused state before use, and comprising 4 electrodes.

FIGS. 2a-2b show schematic views of one embodiment of a lancet needle comprising a catching device, before use (a), and after use (b).

FIGS. 2c-2d show schematic views of another embodiment of a lancet needle comprising a catching device, before use (c), and after use (d).

FIGS. 2e-2f show schematic views of yet another embodiment of a lancet needle comprising a catching device, before use (e), and after use (f).

FIGS. 3a-3b show schematic views of an embodiment of a lancet comprising a protective cap and catch hook, in a resting state (a), and in a used state (b).

FIGS. 3c-3d show schematic views of another embodiment of a lancet comprising a protective cap, seal and catch hook, in a resting state (c), and iii a used state (d).

FIG. 3e shows a side view of an embodiment of a lancet according to the present invention and the contact via a lancet holder, before actuation.

FIG. 3f shows a top view of the lancet of FIG. 3e, after actuation.

FIGS. 4a-4c show schematic views of embodiments of the present invention in which the lancet is used (a) to check the filling level, (b) as a counter electrode, or (c) for a combination of filling level measurement and as a counter electrode (c).

FIGS. 5a-5c show schematic views of different embodiments of chamber geometries as (a) cube shaped, (b) oval whole chamber, and (c) oval semi-chamber.

FIG. 5d shows a side view of the embodiment of FIG. 5c.

FIG. 6 shows a schematic representation of an embodiment of a protective cap having an additional sealing function for a second opening.

In order that the present invention may be more readily understood, reference is made to the following detailed descriptions and examples, which arc intended to illustrate the present invention, but not limit the scope thereof.

### DETAILED DESCRIPTION OF EMBODIMENTS OF THE PRESENT INVENTION

The following descriptions of embodiments are merely exemplary in nature and are in no way intended to limit the present invention or its application or uses.

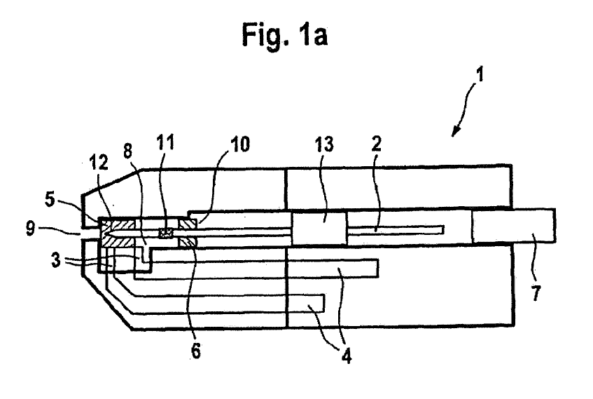

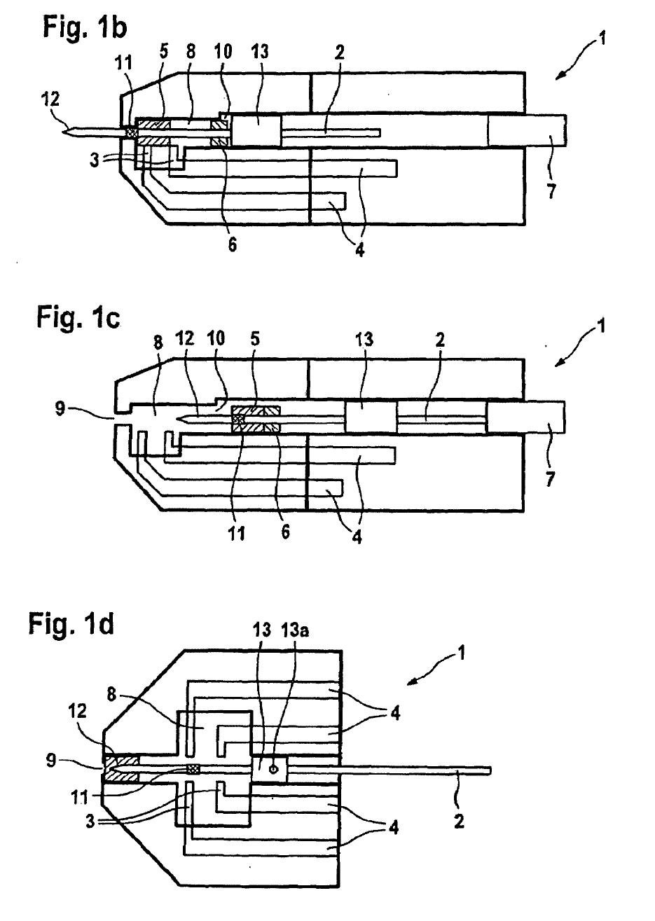

A disposable test element, or dispo, is shown in three different action states according to the embodiment in FIGS. 1a-1c. FIG. 1a shows the dispo (1) with a lancet (2) in an unused state. The tip (12) of the lancet is surrounded by a protective cap (5). The lancet tip (12) is located, together with at least one electrode (3), in a test chamber (8). Contacts (4) connect the electrodes (3) with a measuring device (not shown). The test chamber (8) has at least two openings (9, 10), wherein the first opening (9) is sealed by the protective cap (5) and the second opening (10) is either also sealed by the protective cap (5) or by a further seal (6). This embodiment of a dispo is shown in use in FIG. 1b. One side of the holder (13) has an opening (13a) configured to engage with a lancet drive (not shown here). Lancet (2) then protrudes from dispo (1) through the opening (9). Finally, FIG. 1c shows the embodiment of the lancet (2) after it has been used in the dispo (1). In the process of retracting back within the dispo (1), the lancet (2) has also pulled back the protective cap (5) as well as the seal (6) to such an extent that the opening (9) as well as the opening (10) are opened, or otherwise no longer sealed. In this state the proximal end of the lancet (2) opposite the tip (12) connects with a contact (7).

In another embodiment, a dispo (1) comprising several electrodes (3) is shown in FIG. 1d. In this case there are 4 electrodes but there can also be more. They serve to carry out control measurements or to measure more than one analyte in the sample liquid. In this exemplary embodiment, the geometry of the test chamber is modified such that the lancet holder (13) additionally has an opening (13a) which ensures that the test chamber is vented after actuation of the lancet. In this embodiment the holder (13) has a venting channel from the opening (13a) to the test chamber (not shown here). Before actuation of the lancet (2), the sterility of the test chamber is ensured by a sealing member which is removed from this opening (13a) or otherwise damaged during the actuation (not shown here).

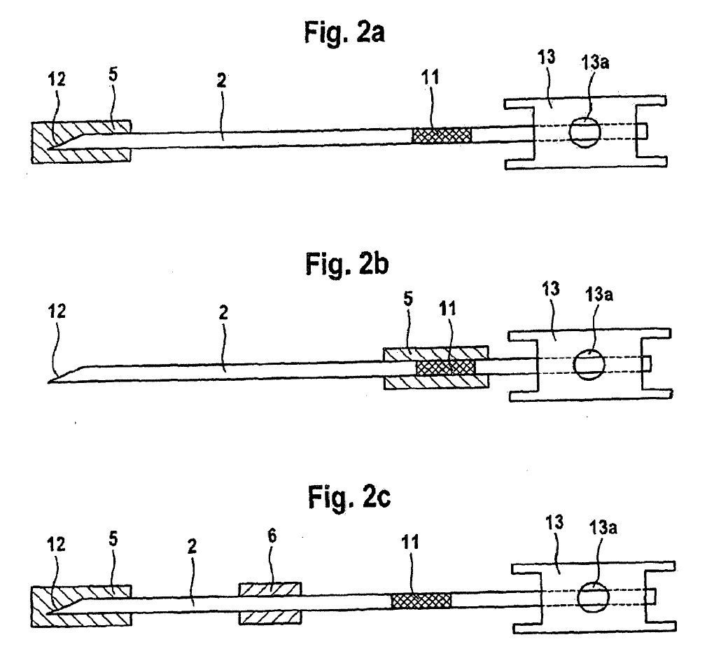

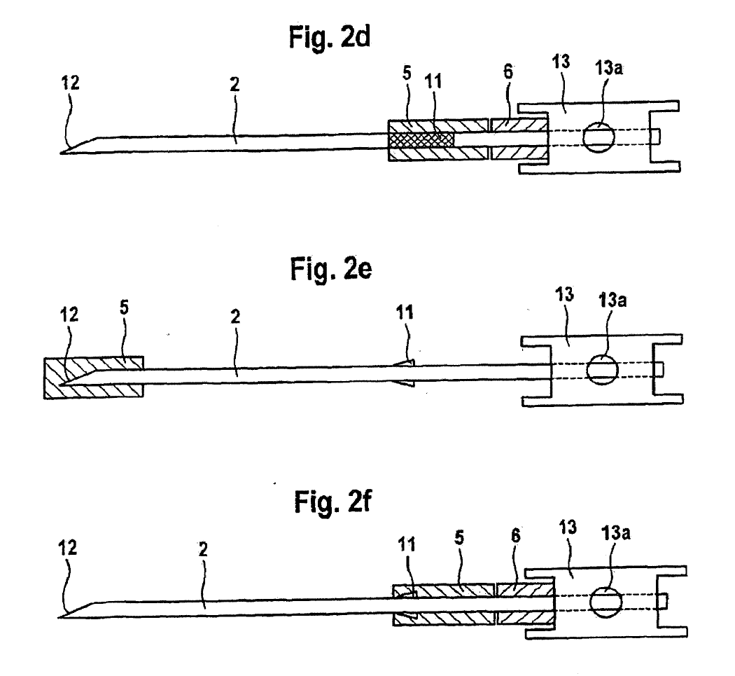

FIGS. 2a-2f show focused views of an embodiment of the lancet (2) in various states of actuation. In this case the principle of a catching device (11) is shown by illustrations of various catching methods. In FIG. 2a the lancet (2) having a catching device (11) is shown in the resting state. The catching device (11) is located between the proximal end and the distal end of the lancet (2). It is configured such that it reaches the protective cap (5) when the lancet (2) is actuated because the protective cap (5) typically has a larger diameter than the housing opening (9) and thus the protective cap (5) is prevented from moving further forwards. As the lancet (2) continues to travel relative to the protective cap (5), the catching device (11) reaches and may surpass the protective cap (5) at the maximum excursion of the lancet (2). When the lancet (2) is retracted, the protective cap (5) is engaged by the catching device (11) and thus is pulled back along with the lancet (2) due to the interaction of the catching device (11) and the protective cap (5). The catching device (11) in one embodiment comprises a roughened surface on which the protective cap (5) as well as, optionally, the second seal (6) are caught due to frictional forces as shown in FIGS. 2a-2d. As described above, the second seal (6) as shown in FIG. 2c is configured to cover second opening (10) of the test chamber before the lancet is actuated.

A further embodiment of the catching device (11) is shown in FIG. 2e. In this case the catching device (11) is in the form of catching bristles.

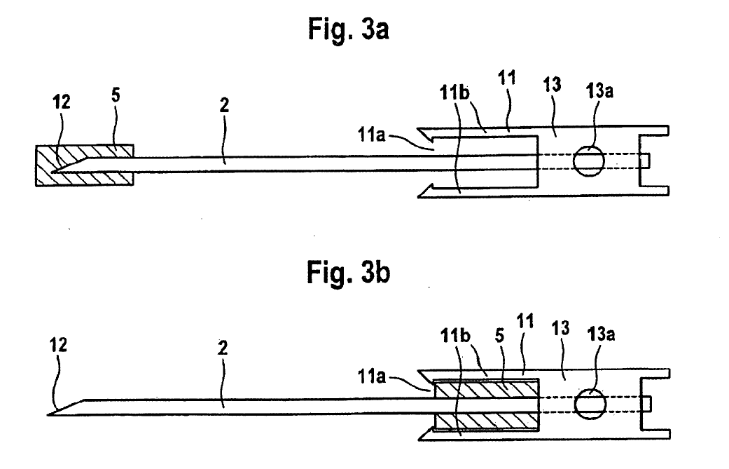

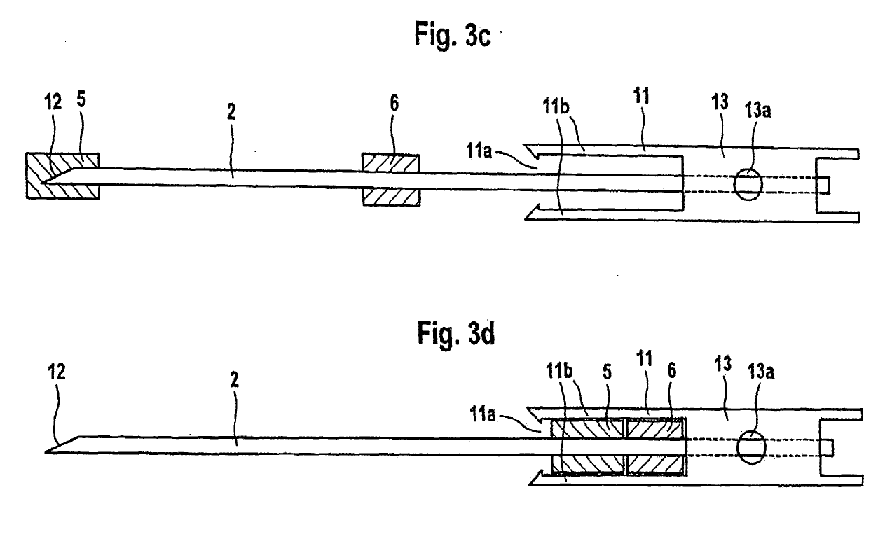

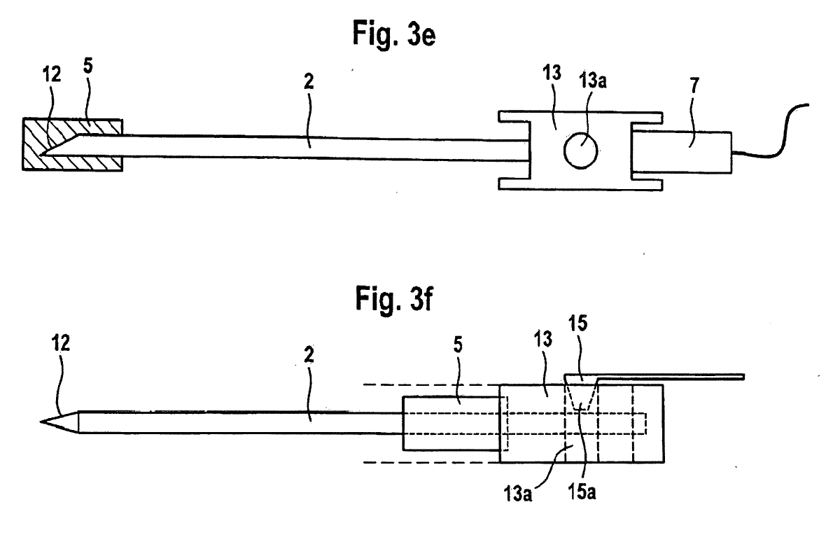

Another embodiment of the catching device (11) is a catch hook as shown schematically in FIG. 3a. The catch hook is attached to the lancet by means of a holder (13) and has an opening (11a) pointing towards the lancet tip (12). The opening (11a) is such that it grips the protective cap (5) at the maximum excursion of the lancet (2) and also transports it back when the lancet (2) is retracted. The catch hook has at least one arm (11b) with a barb on its distal end which slides over the protective cap (5) when the lancet (2) is actuated and thus catches the protective cap (5). Figures protective cap (5). FIGS. 3b and 3d show this catching device after actuation of the lancet (2). In these diagrams the catch hook is equipped with two catching arms (11b) and can thus enclose the protective cap (5) as well as the seal (6) (in embodiments in which this seal is included) from two sides. The catch hook can, however, also have more than 2 catching arms (11b) as desired. In FIGS. 3c and 3d this principle of a catch hook having at least one arm (11b) is shown for the case of the additional seal (6). FIG. 3c shows the lancet in a resting state before actuation and in FIG. 3d the lancet is shown after actuation with a retracted protective cap (5) and seal (6).

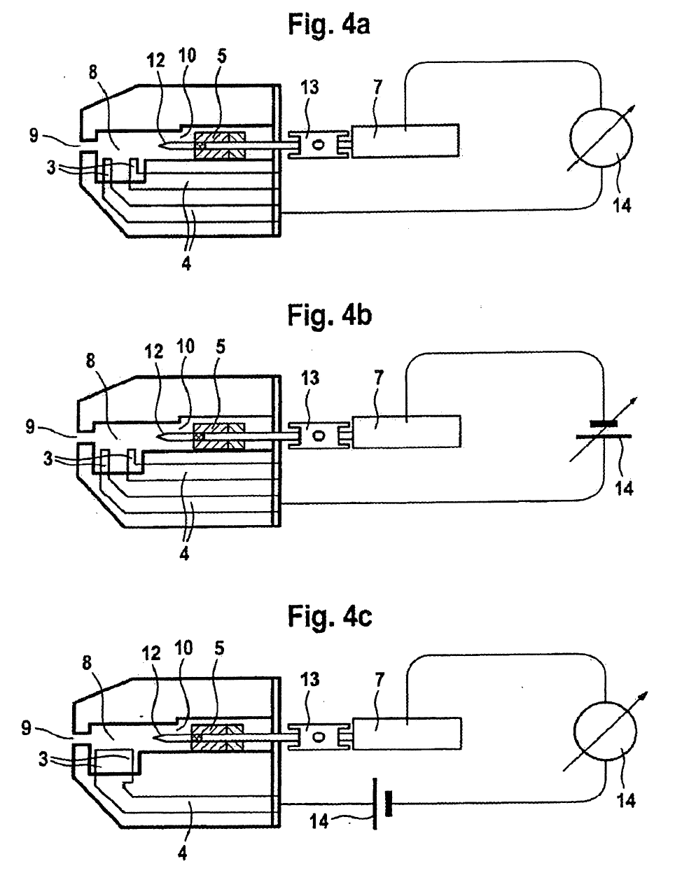

In the embodiment shown in, FIGS. 3e and 3f, after actuation a spring contact (7) engages in an opening (13a) of the lancet holder (13). In this process the bent tip (15a) of the spring contact (7) makes contact with the lancet (2). In such embodiments, the lancet (2) can be further used to check the filling level or as a counter electrode, such as in the configurations of FIGS. 4a-4c. In this case the lancet (2) connects with a spring contact (7) after actuation.

According to the embodiment of FIG. 4a, in order to measure the filling level, the changing potential between or across the lancet (2) and electrode (3) is measured. The filling of the test chamber (8) can also be detected by measuring a current when a direct voltage or alternating voltage is applied between or across lancet (2) and electrode (3). In addition, the lancet needle can also be used as a measuring electrode according to the embodiment shown in FIG. 4b. Also in this case the lancet (2) connects with a contact (7) after it has been retracted and a direct voltage or alternating voltage (14) is applied between or across the electrode (3) in the chamber and the lancet (2). The change of the current flow due to the reaction of the test liquid with the test chemistry on the electrode (3) can be measured in this manner. In this case only one electrode is required in the test chamber. FIG. 4c shows a combination of the two additional properties of the lancet (2), i.e. measurement of the filling level and as a so-called counter or reference electrode. In this case two separate electric circuits (14) are applied to the lancet (2) and electrode (3).

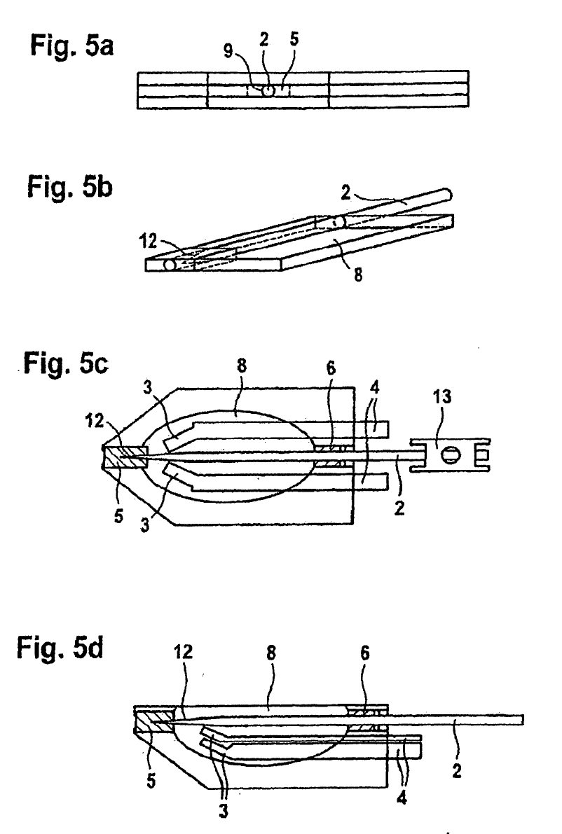

FIG. 5a shows a front view of an embodiment of the dispo (1) in which the protective cap (5) with the lancet (2) is arranged in the opening (9). The path of the lancet (2) in the test chamber (8) can be seen in FIG. 5b which shows the test chamber in a perspective view.

The embodiment of FIG. 5c shows an oval test chamber which extends beyond the electrodes to the protective cap (5). An alternative embodiment of a chamber is shown in FIG. 5d in which the oval chamber is delimited by a boundary on the side opposite to the electrodes thus creating a smaller chamber volume.

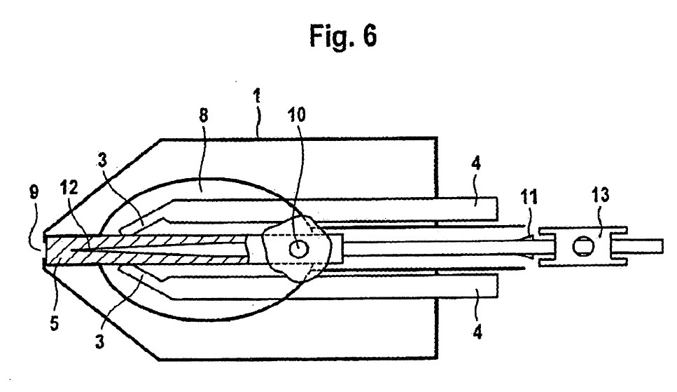

FIG. 6 shows a system in which the protective cap (5) is additionally used as a sealing mechanism for the second opening (10), which generally acts as a venting hole to enable a more efficient filling of the chamber. In this embodiment, second opening (10) is provided in a cover foil overlaying the test chamber (8). In this system as shown, the electrodes (3) which extend into the chamber (8) can be seen as well as the protective cap (5) which seals the chamber and the lancet tip (12) and also encloses a further part of the lancet (2). The lancet holder (13) is located at the proximal end of the lancet (3). The catching device (11) is arranged in front of the lancet holder in the direction of the distal end of the lancet (2).

These various embodiments having been described, it will be appreciated that embodiments according to the present invention generally comprise a dispo in which the three functions lancing, blood transfer from the wound generated by the lancing to the test element and detection of the analyte are integrated in one body. The following description provides additional details for various aspects of the dispo, its integrated functions, and its operation.

The basic body of a protective cap for the analytical device according to the embodiments of the present invention comprises a generally rigid plastic body, the outer shape of which is typically appropriately adapted to the purposes of closing one or more openings. A lancet needle is embedded in this plastic in such a manner that its tip generally does not protrude beyond the front edge of the plastic body. In one embodiment, the protective cap has cross-pieces which are used to lock the needle in position in the plastic body and to guide it during the lancing movement. However, most of the needle is typically not connected to the plastic body in order to minimize the frictional forces during the lancing movement. The contact areas between the needle and protective cap are preferably kept to a minimum and can be pretreated, for example siliconized.

The analytical device generally comprises a housing in which the lancet needle with the protective cap and the test element are located. In the case of electrochemical measurement at least one electrode is typically present in the test chamber. In one embodiment the housing comprises two housing parts. These housing parts can be produced from different plastics by injection molding processes. A non-limiting selection of polymers for such plastics include polyester, polycarbonate, polyvinyl chloride, polymethyl methacrylate, copolyester as well as mixtures thereof. If the test element is evaluated optically, part of the housing is typically made of a transparent material.

The lancets according to the invention are generally designed for single use and can therefore be referred to as single-use blood lancets or disposable blood lancets. The lancet of the present invention comprises a needle (lancet needle) with a tip. The needle usually has a length of from several millimeters (mm) to a few centimeters (cm) and has an elongate shape. Needles typically have a cylindrical design because this needle shape is particularly easy to manufacture; however, needle shapes formed in a different manner are also possible. The tip area of the needle comprises the needle tip which is inserted into tissue during the intended use of the lancet. Hence, the tip of the lancet needle is that part of the lancet which comes into contact with the skin of the individual to be lanced, which punctures the skin and thus results in an outflow of a body fluid such as blood or interstitial fluid.

The tip of the lancet needle can for example be symmetrical with respect to rotation as is in general the case for pins. However, in one embodiment the needle tip comprises one or more ground surfaces. The edges which are formed in this process and are inclined relative to the longitudinal axis of the needle and converge into a tip serve as a sharp cutting edge for the puncture and make the puncture process less painful than is the case with uncut needles.

The lancet needle of the lancet according to embodiments of the invention is typically manufactured from a material which is sufficiently hard to withstand the mechanical stress during the puncture process, the processing steps or possibly other stresses that may occur without deformation. In addition the material should be such that no part can break off or become detached during the puncturing process. Finally it should also be possible to work the needle material in such a manner that the needle tip can be ground to make it sufficiently pointed and optionally also the optionally also the edges of the needle tip to make them sufficiently sharp. Materials that are generally suitable for lancet needles are metals, such as high-grade steels. If the lancet does not have to act as a device to measure the filling level or as a counter electrode, needles made of silicon, ceramics or plastics are also conceivable materials for producing the lancet needle.

In one embodiment at least the tip of the lancet needle of the lancet according to the invention is surrounded according to the invention by the protective cap. In this connection it is important that in the area of the tip of the lancet material, the protective cap comprises a material that can be pierced by the lancet tip. If the protective cap is manufactured from an elastic material in one embodiment, it surrounds the lancet lip generally completely. Thus, the lancet tip is essentially sealed off from the surroundings. The elastic material of the protective cap, which in various embodiments can either completely or only partially form the protective cap, is characterized in that it is soft, deformable and capable of being pierced by the tip of the lancet needle without damaging the tip. In the case of a non-elastic protective cap, the lancet tip is typically surrounded by the protective cap in such a manner that a hollow space is present between the lancet tip and the wall of the protective cap. The wall of the protective cap material is then of such a thickness that also in this case no deformation and wear on the cut edges of the lancet tip occur during the piercing.

During the lancing process, the lancet needle is moved along its longitudinal axis relative to the protective cap and its tip emerges from the housing through the protective cap so that it can puncture the skin of the individual to be examined in order to collect, for example, blood.

The elastic material of the protective cap which generally completely surrounds the tip of the lancet needle ensures the sterility of the lancet needle tip before it is used, until directly before its use. Consequently, in one embodiment, the elastic material is germ-proof with regard to the penetration or escape of germs in the unused state of the lancet needle. Moreover, the elastic material comprises a mechanical protection for the lancet needle tip and thus prevents accidental injury on the lancet needle tip.

Rubber, caoutchouc, silicone, elastomers and in particular thermoplastic elastomers have proven to be suitable as an elastic, material for the embodiments of the protective cap according to the present invention. They generally have important properties for the present invention: they are are soft, deformable, can be pierced by the lancet needle without damaging the tip and seal tightly around the unused lancet needle tip. Furthermore, they can be used for injection molding processes which allows a mass production of lancets in large numbers.

Thermoplastic elastomers which arc also referred to as elastoplasts or thermoplasts or thermoplastic caoutchoucs have, in the ideal case, a combination of the functional properties of elastomers and the processing properties of thermoplasts. Thermoplastic elastomers are for example styrene oligoblock copolymers (so-called TPE-S), thermoplastic polyolefins (TPE-O), thermoplastic polyurethanes (TPE-U), thermoplastic copolyesters (TPE-E) and thermoplastic copolyamides (TPE-A). In particular thermoplastic elastomers based on styrene-, ethylene-butylene-styrene-polymers (SEBS polymers, e.g. Evoprene® from Evode Plastics or Thermolast K from Gummiwerk Kraiburg GmbH) have for example proven to be suitable.

During the lancing process, the lancet needle is moved relative to the protective cap. During this process the protective cap is typically locked in position by the lancing aid or the lancing device. The lancet needle can be specially shaped for the purposes of its propulsion, for example it can have a needle head at the end opposite to the tip, or in addition to the protective cap which surrounds the tip, it can have a further lancet body or the aforementioned lancet holder which can be gripped by a drive element of the lancing aid. The shaped element of the needle or of the additional lancet holder can interact in a suitable manner with a corresponding drive device in the lancing device (lancing aid). In general such means can be referred to as a drive device of the needle. Such drive devices are sufficiently known to a person skilled in the art for example from U.S. Pat. No. 6,783,537B1 or EP 1 336 375, the disclosures of which are hereby incorporated herein by reference in their entireties.

In order to increase the stability of the elastic material, it is possible to combine it with a different material, for example a stiff plastic material. The outside of the elastic material which does not come into contact with the tip of the lancet needle can for example be stabilized with a layer of a stiff material, for example of a stiff plastic. It is also possible to manufacture the lancet protection from an elastic material only in the area of the lancet needle tip and to manufacture the remainder of the lancet cover from conventional stiff plastics. In this connection the elastic material and the stiff material can be glued together or joined together by an injection molding process for process for example a two-component injection molding process. The stiff material of the lancet cover ensures a mechanical stabilization of the elastic material during the lancing process and facilitates the immobilization of the elastic part of the protective cap by the lancing aid during the lancing process. The stiff material can also be part of a test element, for example a capillary gap test element as described in WO 99/29429, the disclosure of which is hereby incorporated herein by reference in its entirety. In another embodiment the whole protective cap can comprise stiff plastic material.

During use, the patient typically does not come into contact or only partially comes into contact with the protective cap during the lancing process. Rather the patient places his finger on the first opening of the housing which is typically not located on the same side as the drive nor where the dispo is coupled to the measuring device. After placing his finger, the patient can trigger a mechanism which moves the lancet from a resting state into an actuation state and returns it to the resting state. In this process the distal end, i.e. the tip of the lancet, moves out of the housing through the opening for a short period. On its way back into the housing, as has been previously described, it also pulls back the protective cap by means of a catching device or other means which is coupled to, connected to or otherwise provided on the lancet body, wherein the protective cap remains in the housing also during the lancing.

Generally, blood is transferred in embodiments according to the invention from the wound/puncture site of the lancet to the measuring site in the following manner: The blood transfer is typically accomplished as it were “automatically” without the help of the user of the dispo according to the invention. For this purpose the dispo can have means for sample liquid transport. These means are typically capillary-active and are for example in the form of a gap or channels in a rigid base body or of absorbent matrix materials. It is also possible to combine these two basic examples, for example, in that the blood is firstly guided through a capillary channel, is received by an absorbent matrix material and is delivered into a test chamber.

Absorbent fleeces, papers, wicks or fabrics have proven to be suitable in the sense of this invention as absorbent matrix materials.

In an alternative embodiment, the body of the analytical device, typically the test chamber, comprises the means for sample liquid transport. As already described, this can be an absorbent wick which is recessed into the chamber or a formed capillary gap which also provides an exit opening for the lancet. As a result the dispo does not have to be moved after the lancing function for the blood collection. In embodiments including a capillary gap, the geometry of the inlet opening can be designed as a funnel or notch such that it facilitates the capture and entry of the blood drop that is generated. The capillary action then ensures that the required amount of blood which can be considerably below 1 microliter, is sucked in. In this manner the blood reaches the test field in the chamber and there it reacts with the test chemistry to generate an electrical signal or a change in color that can be analyzed. The capillary gap can be molded into the plastic during the injection molding or can be subsequently introduced into the plastic body for example by stamping or milling. The sample drop is typically sucked into the capillary channel by means of the fact that the area that is exposed by the recess is hydrophilized and directly borders a capillary-active zone at least in the direction of the capillary transport channel.

In this regard, hydrophilic surfaces are surfaces which attract wafer. Aqueous samples, also including blood, spread well on such surfaces. Such surfaces are characterized among others in that at the interface a water drop forms an acute boundary or contact angle on these surfaces. In contrast, an obtuse boundary angle is formed at the interface between the water drop and the surface on hydrophobic surfaces, i.e. surfaces which repel water. The ability of a capillary to suck up a liquid correlates with the wettability of the channel surface with the liquid. In the case of aqueous samples, this means that a capillary should be manufactured from a material whose surface tension approximates that of water (72 mN/m) or exceeds this value.

Sufficiently hydrophilic materials for constructing a capillary which rapidly sucks up aqueous samples are for example glass, metals or ceramics. However, these materials are less suitable for use in test carriers because they have serious disadvantages. This is, for example, the risk of breakage in the case of glass or ceramics or a change in the surface properties with time in the case of numerous metals. Therefore plastic foils or plastic molded parts are usually used to manufacture test elements. The plastics that are used as a rule hardly exceed a surface tension of 45 mN/m. Even with the most hydrophilic plastics, from a relative point of view, such as for example example polymethyl methacrylate (PMMA) or polyamide (PA) it is only possible, if at all, to construct capillaries that suck very slowly. Capillaries made of hydrophobic plastics such as for example polystyrene (PS), polypropylene (PP) or polyethylene (PE) aspirate essentially no aqueous samples. This means that plastics have to be made hydrophilic i.e. hydrophilized in order to be used as a construction material for test elements with capillary-active channels.

The surface of the capillary channel is ideally hydrophilized by using a hydrophilic material for its manufacture which, however, is not able to itself absorb the sample liquid or only to an insubstantial extent. In cases where this is not possible, a hydrophobic or only very slightly hydrophilic surface can be hydrophilized by a suitable coating with a stable hydrophilic layer that is inert towards the sample material for example by covalently binding photoreactively equipped, hydrophilic polymers to a plastic surface by applying layers containing wetting agents or by coating surfaces with nanocomposites by means of sol-gel technology. Furthermore, it is possible to increase the hydrophilicity of a surface by thermal, physical or chemical treatment of the surface. This for example also includes the plasma treatment of a surface. This is for example carried out using high-energy oxygen or other polarizing media.

The hydrophilization in one embodiment of the present invention is accomplished by using thin layers of oxidized aluminum. These layers are either directly applied to the desired components of the test element for example by vacuum coating the workpieces with metallic aluminum by evaporation and subsequently oxidizing the metal, or they are used in the form of metal foils or metal-coated plastic foils to construct the test carrier where said foils also have to be oxidized to achieve the desired hydrophilicity. In this regard, metal coat thicknesses of about 1 to about 500 nm are sufficient. The metal coat is subsequently oxidized to form the oxidized form where in addition to electrochemical, anodic oxidation, especially oxidation in the presence of water vapor or by boiling in water have proven to be particularly suitable methods. In one embodiment, the oxide layers achieved in this manner have thicknesses between about 0.1 and about 500 nm. In other embodiments, the thickness is between about 10 and about 100 nm. Larger layer thicknesses of the metal layer as well as of the oxide layer can in principle be achieved in practice but do not exhibit any further advantageous effects.

In one embodiment, the detection element of the analytical test element according to the invention contains all reagents necessary for the detection reaction of the target analyte in the sample and optionally auxiliary substances. The detection element can also contain only some of the reagents or auxiliary substances. Such reagents and auxiliary substances are very well-known to a person skilled in the art who is familiar with the technology of analytical test elements or diagnostic test carriers. The detection element can for example contain enzymes, enzyme substrates, indicators, buffer salts, inert fillers and such like for analytes that are to be detected enzymatically. The detection element can comprise one or more layers and optionally contain an inert support on the side of the detection element which is not brought into contact with the sample. For embodiments in which the detection reaction results in an observable change in color, which in this context is typically understood as a change in color, the formation of a color or the disappearance of color, suitable measures should be employed to ensure that the support allows a visual or optical observation of the detection reaction. For this purpose the support material of the detection element can itself be transparent and for example have a transparent plastic foil such as for example a polycarbonate foil or a transparent opening on the detection side. In addition to detection reactions which lead to color changes, a person skilled in the art also knows other principles of detection which can be achieved with the described test elements such as electrochemical sensors.

In the embodiments configured for electrochemical detection of the analyte, at least one electrode is located in the test chamber. The electrode is connected to the measuring device by a contact on the dispo. The lancet can be used in this case as a second electrode. The lancet in one embodiment is connected to the measuring device by a spring contact, but rigid contacts of the lancet with the measuring device are also possible for other embodiments. The measurement can be carried out with direct current as well as with alternating current. It is also possible to use the lancet to check the filling level. A spring contact can also be used for this purpose which, after the lancet has been used, connects the lancet with the measuring device and a change in the current strength or voltage occurs when the lancet makes contact with the liquid as it enters.

The test chamber contains the lancet tip with protective cap and the necessary detection chemicals and electrodes in the case of an electrochemical detection. The size of the test chamber is such that a minimum volume of test liquid is required. In one embodiment, this is less than about 1 μL. Hydrophilic materials are typically used to manufacture the test chamber for an optimal transport of the liquid into the test chamber through the outlet opening.

The test chamber can have various geometries. Thus, the chamber can be cube-shaped or cuboid-shaped. The chamber can, however, also adopt the shape of a round or oval hemisphere. The chamber has at least one opening through which the lancet exits when actuated. In the resting state before use this opening is kept closed by the protective cap which surrounds the lancet tip. After the lancet is actuated, a further venting of the chamber is required in order to achieve a capillary effect into the chamber. This can be provided by a second opening of the chamber or by a vent hole in the lancet holder which is sealed before use of the lancet by a foil or other sealing. The geometry and the volume of the chamber depend on the number of electrodes in the chamber. The number of electrodes depends on how the device is used. For an electrochemical analysis at least one electrode is in the test chamber and the lancet can be used as a counter electrode or reference electrode. This can be complemented by further electrodes. The addition of further electrodes allows more than one analyte to be determined independently of one another. These analytes can for example be blood constituents such as cholesterol, triglycerides, coagulation factors and other blood parameters.

In one embodiment, the volume of the chamber is between about 100 nl and about 1000 nl but in other embodiments it can be larger when using a plurality of electrodes. In other embodiments, the volume is between about 300 nl and about 600 nl. In yet other embodiments, the volume is about 500 nl. The electrodes and their contacts are made of a conductive material such as conductive plastics or metal. When the lancet tip is used as an electrode, the electrodes and their contacts and also the lancet comprise a conductive material such as e.g. aluminum, lead, iron, gallium, gold, indium, iridium, carbon (such as graphite), cobalt, copper, magnesium, nickel, niobium, osmium, palladium, platinum, mercury (such as amalgam), rhenium, rhodium, selenium, silicon (such as highly-doped polycrystalline silicon), silver, tantalum, titanium, uranium, vanadium, tungsten, tin, zinc, zirconium, mixtures and alloys thereof, oxides or metal mixtures of mixtures of the listed elements. The contacts and electrodes typically comprise one of gold, platinum, palladium, iridium or mixtures or alloys of these metals. In this connection the contacts can be made of a different material than the electrodes. Equally the lancet tip can have a different composition than the remainder of the lancet body. In one embodiment, a silver/silver halogenide electrode is provided as a reference electrode and a (e.g. screen printed) graphite electrode is provided as the working electrode.

When the chemical reaction in the test chamber is detected optically, the housing of the device typically has at least one optically transparent housing wall which is mounted either below or above the test chemistry. If only optical measurements are carried out, no electrodes have to be present in the test chamber, but a combination of electrochemical and optical measurement is also possible. For embodiments configured for optical detection, a light beam is aligned onto the test field and its interaction with the liquid can either be measured in reflection or transmission. In the case of a transmission measurement, an optically transparent housing wall is used on the side of the irradiation as well as on the detection side. In the case of an optical detection, a detection element which is a component of the test element and contains the detection reagents is attached to the transparent surface of the test chamber. When the test liquid reacts with the test reagent in the detection element, an optical feature is changed which can be detected with the aid of an optical module. This optical module can be a photosensor or a photomultiplier or any other optical sensor unit known from the prior art. The source of radiation can also be one that is known in the prior art. In the case of an optical measurement the retracted lancet can also be used as a measuring device for the filling level.

The catching device which is used to pull back the protective cap and/or another seal can have various embodiments. One embodiment according to the invention is to roughen the lancet surface at a suitable site on the lancet body. As a result a sufficient rubbing action is created when this roughened surface makes contact with the sealing body or sealing bodies such that the sealing body or sealing bodies adhere thereto when the lancet is retracted.

A second embodiment according to the invention is a catch hook which in turn various embodiments can have. The principle is that the sealing body hooks on the barb of the lancet body when the lancet emerges to its maximum extent. This allows the sealing body or sealing bodies to bodies to be pulled back with the lancet. In this connection the catch hook can comprise bristle-shaped elements or hooks extending from the lancet which are aligned towards the proximal end of the lancet. These hooks can be made of various materials such as metal, ceramics or polymers.

Another embodiment for the catching device is a catch hook which slides over and encloses the sealing body. The clasping of the sealing body allows the seals to be pulled back when the lancet is retracted again after the actuation. This catch hook is connected to the lancet body by means of a holder. The catch hook has at least one arm which extends towards the lancet tip and is equipped with one hook at its distal end. The hook slips over the sealing body or sealing bodies at the maximum excursion of the lancet. In one embodiment, at least two arms are provided in order to ensure that the seals are captured.

This catch hook or the roughened surface of the lancet pulls the protective cap as well as other seals that may be present into the housing. The retracted seals open the exit opening as well as a further opening for venting the test chamber. This sealing of the test chamber also prevents contamination of the electrodes and test reagents during storage. Furthermore, the protective cap prevents an accidentally premature contact of the patient with the lancet.

In general the function of the typical embodiments according to the present invention is as follows:

The dispo is inserted into the receiving device of a (blood sugar) measuring device and is thus immobilized. In the case of dispos that are stored in a magazine, a magazine of dispos is inserted into the measuring device. The drive mechanism of the lancing unit is tensioned and coupled to the drive device of the dispo. When the dispo is fixed in position in the measuring device, the contacts of the dispo make contact with the electrical supply in the measuring device. The user contacts the opening of the analytical device with his finger or a site on the body at which it is intended to carry out the measurement. When the lancing process is triggered, the needle is moved forwards and in doing so exits at high speed from the opening through the protective cap. The entire lancing process occurs within a few milliseconds. After the skin has been punctured the needle is retracted again. In this process a catching device which is located on the lancet pulls along the protective cap and optionally additional seals. The drive is optionally decoupled again. If the lancet is used as a counter electrode, the lancet is connected to an additional spring contact which additional spring contact which can be integrated into the drive unit. The user (additionally) contacts the opening of the device with his collection device so that the suction opening (e.g. capillary) cap take up a drop of blood. The suction action of the means for sample liquid transport transports the blood in the dispo to a site in the test chamber at which a test element comprising a detection element is located. A reaction takes place in the detection element between the blood components to be detected and the detection reagents which is for example detected by means of photometric or electrochemical detection. A measured result is calculated from the electronic data and displayed optically or acoustically to the user. In the case of dispos stored in a magazine, the magazine is advanced by one step. In the case of single-use dispos, the used dispo is ejected or removed by hand.

Measuring devices which are known to a person skilled in the art which have various features such as algorithms for the evaluation and various energy sources for supplying the dispo are described in the following references: U.S. Pat. No. 4,963,814; U.S. Pat. No. 4,999,632; U.S. Pat. No. 4,999,582; U.S. Pat. No. 5,243,516; U.S. Pat. No. 5,352,351; U.S. Pat. No. 5,366,609; U.S. Pat. No. 5,405,511; U.S. Pat. No. 5,438,271; the disclosures of which are hereby incorporated herein by reference in their entireties.

A disposable according to the invention can in principle be manufactured by the following simple steps: injection-molding the housing parts; injection-molding the plastic body (protective cap) including embedding; the lancet needle (optionally while generating the “needle head”, i.e. the thickening which can be gripped by a lancing device); extrusion coating the needle tip with soft plastic; sterilizing the “crude dispos” for example by means of ionizing radiation (these “crude dispos” can be preferably present as tape material which is divided into individual dispos for example by cutting or punching); mounting (sputtering, printing etc.) or embedding (laser ablation, etching, injection molding etc.) the electrodes in the housing; introducing the test element comprising a detection element into the housing on the electrodes; assembling i.e. connecting the “crude dispos” with the housing; and sealing and packaging the housing.

Irrespective of whether the “crude dispos” according to the invention are manufactured as roll or tape material in a continuous process or batchwise or individually, the lancet and housing can be connected together before or after sterilization of the lancet. A sterilization after assembly requires that the test chemistry be sufficiently covered during the sterilization because otherwise the the reagents may be damaged.

Another aspect of the present invention is the use of a plastic material as a component of a lancet of an analytical device where the plastic material is used to maintain the sterility of at least the tip of a lancet needle in the unused state.

The use according to the invention of an elastic material to screen the tip of the lancet needle ensures the sterility of an unused lancet needle tip.

In embodiments in which a sample of biological fluid is obtained directly from a person or animal, the lancet needle tip should be made sterile in the unused state by means of suitable measures such as for example treatment with ionizing radiation. Once sterilized, the lancet needle tips remain sterilized by means of the respective protective caps which inter alia comprise an elastic material.

The use according to the invention of the protective cap additionally enables at least one opening of the test chamber to be sealed. This at least one opening is also the exit opening for the lancet and the inlet opening for the body fluid.

The features disclosed in the above description, the claims and the drawings may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. An analytical device comprising a lancet and a test element in an integrated arrangement, the lancet comprising a lancet needle with a tip and a protective cap which generally completely surrounds the lancet needle at least in the area of the tip, wherein the lancet needle can be displaced in at least one direction relative to the protective cap, and wherein the lancet needle is configured to engage the protective cap after displacement of the lancet needle in the at least one direction and thereafter to pull the protective cap during displacement of the lancet needle in a retracting direction, the test element comprising a chamber containing a reagent system, the chamber having a first opening configured to be generally sealed by the protective cap prior to and during displacement of the lancet needle in the at least one direction and configured thereafter to be generally opened upon displacement of the lancet needle in the retracting direction.

2. The analytical device according to claim 1 wherein the protective cap comprises an elastomer.

3. The analytical device according to claim 1 wherein the protective cap encloses the needle tip in a sterile manner.

4. The analytical device according to claim 1 wherein the analytical device comprises structural means for transporting sample liquid.

5. The analytical device according to claim 4 wherein the structural means for transporting sample liquid comprises a structure selected from the group consisting of a capillary gap, a capillary channel, a wick comprising an absorbent material, and a cross-piece made of an absorbent material.

6. The analytical device according to claim 1 wherein the chamber further comprises a second opening and wherein the lancet further comprises a seal configured to generally seal the second opening prior to displacement of the lancet needle in the at least one direction.

7. The analytical device according to claim 6 wherein the protective cap comprises the seal.

8. The analytical device according to claim 6 wherein the lancet further comprises a catching device provided on the lancet needle, the catching device being configured to engage and pull the protective cap when the lancet needle is displaced in the retracting direction, and wherein when the lancet needle is displaced in the retracting direction, the protective cap in turn pulls the seal along with it and opens the second opening, wherein the second opening comprises an air vent.

9. The analytical device according to claim 1 wherein the lancet further comprises a catching device provided on the lancet needle, the catching device being configured to engage and pull the protective cap when the lancet needle is displaced in the retracting direction.

10. The analytical device according to claim 1 wherein upon complete displacement of the lancet needle in the retracting direction, the lancet is configured for electrical connection to a measuring device wherein the tip then comprises an electrode to be used for at least one of measuring the level of sample liquid and serving as a counter electrode of the analytical device.

11. A method for producing an analytical device comprising the steps of: providing a lancet comprising a lancet needle and a protective cap, the lancet needle having a tip located in the protective cap the lancet further comprising a catching device provided on the lancet needle configured to pull the protective cap back along with the needle when the lancet is displaced in a retracting direction; providing a test element comprising a chamber containing a reagent system and an opening in the chamber; sterilizing the lancet; and introducing the lancet into the chamber such that the protective cap seals the opening of the chamber.

12. An analytical system comprising the analytical device of claim 1, the analytical device further comprising a reagent system disposed within the chamber, the analytical system further comprising a detection unit configured to engage the analytical device and to detect signals generated during a reaction of the reagent system with an analyte in a sample introduced into the chamber, and an evaluation device configured to determine a concentration of the analyte from the signals.
